(19) United States
(12) Patent Application Publication
(10) Pub. No.: US 2022/0257529 A1
(43) Pub. Date: Aug. 18, 2022

(54) USE OF CANNABIDIOL IN THE TREATMENT OF EPILEPTIC SPASMS

(71) Applicant: GW Research Limited, Cambridge (GB)

(72) Inventors: Geoffrey GUY, Cambridge (GB); Volker KNAPPERTZ, Cambridge (GB); Eduardo DUNAYEVICH, Cambridge (GB); Daniel CHECKETTS, Cambridge (GB)

(21) Appl. No.: 17/611,824

(22) PCT Filed: May 15, 2020
(86) PCT No.: PCT/GB2020/051185

§ 371 Date: Nov. 16, 2021

(30) Foreign Application Priority Data
May 23, 2019 (GB) 1907283.4

(51) Int. Cl. A61K 31/05; A61K 31/4015; A61K 31/5513; A61K 31/197; A61K 31/423; A61K 31/4192; A61K 31/165; A61K 31/36; A61K 31/53; A61P 25/08

(52) U.S. Cl. CPC A61K 31/05; A61K 31/4015; A61K 31/5513; A61K 31/197; A61P 25/08; A61K 31/4192; A61K 31/165; A61K 31/36; A61K 31/53; A61K 31/423

(57) ABSTRACT

The present invention relates to the use of cannabidiol (CBD) for the treatment of epileptic spasms. In particular the epileptic spasms that are treated are associated with Tuberous Sclerosis Complex (TSC). In a further embodiment the epileptic spasm is treated with CBD in combination with clobazam. Preferably the CBD used is in the form of a botanically derived purified CBD which comprises greater than or equal to 98% (w/w) CBD and less than or equal to 2% (w/w) of other cannabinoids. The other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w). The botanically derived purified CBD preferably also comprises a mixture of both trans-THC and cis-THC. Alternatively, a synthetically produced CBD is used. Where the CBD is given concomitantly with one or more other anti-epileptic drugs (AED) including clobazam, the CBD may be formulated for administration separately, sequentially or simultaneously with one or more AED or the combination may be provided in a single dosage form.

### FIELD OF THE INVENTION

The present invention relates to the use of cannabidiol (CBD) for the treatment of epileptic spasms. In particular the epileptic spasms that are treated are associated with Tuberous Sclerosis Complex (TSC). In a further embodiment the epileptic spasm is treated with CBD in combination with clobazam.

Preferably the CBD used is in the form of a botanically derived purified CBD which comprises greater than or equal to 98% (w/w) CBD and less than or equal to 2% (w/w) of other cannabinoids. The other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w). The botanically derived purified CBD preferably also comprises a mixture of both trans-THC and cis-THC. Alternatively, a synthetically produced CBD is used.

Where the CBD is given concomitantly with one or more other anti-epileptic drugs (AED) including clobazam, the CBD may be formulated for administration separately, sequentially or simultaneously with one or more AED or the combination may be provided in a single dosage form.

### BACKGROUND TO THE INVENTION

Epilepsy occurs in approximately 1% of the population worldwide, (Thurman et al., 2011) of which 70% are able to adequately control their symptoms with the available existing anti-epileptic drugs (AED). However, 30% of this patient group, (Eadie et al., 2012), are unable to obtain seizure freedom from the AED that are available and as such are termed as suffering from intractable or “treatment-resistant epilepsy” (TRE).

Intractable or treatment-resistant epilepsy was defined in 2009 by the International League Against Epilepsy (ILAE) as “failure of adequate trials of two tolerated and appropriately chosen and used AED schedules (whether as monotherapies or in combination) to achieve sustained seizure freedom” (Kwan et al., 2009).

Individuals who develop epilepsy during the first few years of life are often difficult to treat and as such are often termed treatment resistant. Children who undergo frequent seizures in childhood are often left with neurological damage which can cause cognitive, behavioral and motor delays.

Childhood epilepsy is a relatively common neurological disorder in children and young adults with a prevalence of approximately 700 per 100,000. This is twice the number of epileptic adults per population.

When a child or young adult presents with a seizure, investigations are normally undertaken in order to investigate the cause. Childhood epilepsy can be caused by many different syndromes and genetic mutations and as such diagnosis for these children may take some time.

The main symptom of epilepsy is repeated seizures. In order to determine the type of epilepsy or the epileptic syndrome that a patient is suffering from an investigation into the type of seizures that the patient is experiencing is undertaken. Clinical observations and electroencephalography (EEG) tests are conducted and the type(s) of seizures are classified according to the ILEA classification.

Generalized seizures, where the seizure arises within and rapidly engages bilaterally distributed networks, can be split into six subtypes: tonic-clonic (grand mal) seizures; absence (petit mal) seizures; clonic seizures; tonic seizures; atonic seizures and myoclonic seizures.

Focal (partial) seizures where the seizure originates within networks limited to only one hemisphere, are also split into sub-categories. Here the seizure is characterized according to one or more features of the seizure, including aura, motor, autonomic and awareness/responsiveness. Where a seizure begins as a localized seizure and rapidly evolves to be distributed within bilateral networks this seizure is known as a bilateral convulsive seizure, which is the proposed terminology to replace secondary generalized seizures (generalized seizures that have evolved from focal seizures and are no longer remain localized).

Focal seizures where the subject's awareness/responsiveness is altered are referred to as focal seizures with impairment and focal seizures where the awareness or responsiveness of the subject is not impaired are referred to as focal seizures without impairment.

In 2001, the International League Against Epilepsy (ILAE) classified epileptic spasms (ES) as a distinct type of epilepsy. Epileptic spasms, also known as infantile spasms, juvenile spasm or West syndrome, are paroxysmal seizures with motoric manifestations consisting of abrupt flexion, extension or combination of flexion/extension of the axial or limb muscles.

The age of onset of ES is typically between four and six months of age; however, ES can occur at any time in the first two years of life and can persist into adulthood. In symptomatic ES, which comprises 45-61% of cases, the etiology can be attributed to an underlying neurological condition of which chromosomal anomalies (8%), cortical malformations (8%), and tuberous sclerosis complex (TSC) (7%) are among the most frequent.

The prognosis of ES is often very poor, with 70-90% of patients showing cognitive impairment and 50% of children developing intractable seizures.

Adrenocorticotropin hormone (ACTH), prednisone, and vigabatrin (VGB) are considered the preferred first-line treatments by different consensus panels and committees. While these medications can be effective at controlling seizures—with responder rates ranging from, 42% to 100% for ACTH, 29% to 59% for prednisone, and 0% to 100% for VGB, they have significant risks, including increased risk of infection for ACTH and prednisone, and visual field defects for VGB.

Cannabidiol (CBD), a non-psychoactive derivative from the cannabis plant, has demonstrated anti-convulsant properties in several anecdotal reports, pre-clinical and clinical studies both in animal models and humans. Three randomized control trials showed efficacy of the purified pharmaceutical formulation of CBD in patients with Dravet and Lennox-Gastaut syndrome.

Based on these three trials, a botanically derived purified CBD preparation was approved by FDA in June 2018 for the treatment of seizures associated with Dravet and Lennox-Gastaut syndromes.

The applicant has found by way of an open label, expanded-access program that treatment with CBD resulted in a significant reduction in epileptic spasms. In addition, there was a significant improvement in cognitive and behavioural domains within this treatment group. There was a particular improvement in patients with TSC who had epileptic spasms.

Furthermore, all patients in the program were taking clobazam and all either had their dose of clobazam reduced or clobazam was discontinued.

### BRIEF SUMMARY OF THE DISCLOSURE

In accordance with a first aspect of the present invention there is provided a cannabidiol (CBD) preparation for use in the treatment of epileptic spasms (ES).

Preferably the CBD preparation comprises greater than or equal to 98% (w/w) CBD and less than or equal to 2% (w/w) other cannabinoids, wherein the less than or equal to 2% (w/w) other cannabinoids comprise the cannabinoids tetrahydrocannabinol (THC); cannabidiol-C1 (CBD-C1); cannabidivarin (CBDV); and cannabidiol-C4 (CBD-C4), and wherein the THC is present as a mixture of trans-THC and cis-THC.

Preferably the CBD preparation is used in combination with one or more concomitant anti-epileptic drugs (AED).

Preferably the one or more AED is selected from the group consisting of: valproic acid, levetiracetam, clobazam, vigabatrin, zonisamide, rufinamide, lacosamide, topiramate and lamotrigine. More preferably the AED is clobazam.

In one embodiment the CBD is present is isolated from cannabis plant material. Preferably at least a portion of at least one of the cannabinoids present in the CBD preparation is isolated from cannabis plant material.

In a further embodiment the CBD is present as a synthetic preparation. Preferably at least a portion of at least one of the cannabinoids present in the CBD preparation is prepared synthetically.

Preferably the dose of CBD is greater than 5 mg/kg/day. More preferably the dose of CBD is 20 mg/kg/day. More preferably the dose of CBD is 25 mg/kg/day. More preferably the dose of CBD is 50 mg/kg/day.

In a further embodiment the epileptic spasms are associated with tuberous sclerosis complex (TSC).

In accordance with a second aspect of the present invention there is provided a method of treating a patient suffering from epileptic spasms (ES) comprising administering a cannabidiol (CBD) preparation to the subject in need thereof.

### DEFINITIONS

Definitions of some of the terms used to describe the invention are detailed below:

Over 100 different cannabinoids have been identified, see for example, Handbook of Cannabis, Roger Pertwee, Chapter 1, pages 3 to 15. These cannabinoids can be split into different groups as follows: Phytocannabinoids; Endocannabinoids and Synthetic cannabinoids (which may be novel cannabinoids or synthetically produced phytocannabinoids or endocannabinoids).

“Phytocannabinoids” are cannabinoids that originate from nature and can be found in the cannabis plant. The phytocannabinoids can be isolated from plants to produce a highly purified extract or can be reproduced synthetically.

“Highly purified cannabinoids” are defined as cannabinoids that have been extracted from the cannabis plant and purified to the extent that other cannabinoids and non-cannabinoid components that are co-extracted with the cannabinoids have been removed, such that the highly purified cannabinoid is greater than or equal to 95% (w/w) pure.

“Synthetic cannabinoids” are compounds that have a cannabinoid or cannabinoid-like structure and are manufactured using chemical means rather than by the plant.

Phytocannabinoids can be obtained as either the neutral (decarboxylated form) or the carboxylic acid form depending on the method used to extract the cannabinoids. For example, it is known that heating the carboxylic acid form will cause most of the carboxylic acid form to decarboxylate into the neutral form.

“Treatment-resistant epilepsy” (TRE) or “intractable epilepsy” is defined as per the ILAE guidance of 2009 as epilepsy that is not adequately controlled by trials of one or more AED.

“Epileptic spasm”, “infantile spasm”, “juvenile spasm” or “West syndrome” is defined as sudden flexion, extension or mixed flexion-extension of proximal and truncal muscles, lasting 1-2 seconds. Spasms typically occur in a series, usually on wakening. Subtle forms may occur with only chin movement, grimacing, or head nodding. Spasms may be bilaterally symmetric, asymmetric, or unilateral, depending on whether they are generalised onset or focal onset.

“Focal Seizures” are defined as seizures which originate within networks limited to only one hemisphere. What happens during the seizure depends on where in the brain the seizure happens and what that part of the brain normally does.

“Focal seizure with impairment” has replaced the term “complex partial seizure”. These seizures usually start in a small area of the temporal lobe or frontal lobe of the brain and involve other areas of the brain within the same hemisphere that affect alertness and awareness. Most subjects experience automatisms during a focal seizure with impaired consciousness.

“Mixed seizures” are defined as the existence of both generalised and focal seizures in the same patient.

The terms “50% responder” and “50% reduction in seizure” are both terms used in clinical studies. In the present application the terms define the percentage of subjects that experienced a greater than or equal to 50% reduction in the total number of seizures during treatment with CBD in comparison to the number experienced during the baseline period before the CBD was administered.

### DETAILED DESCRIPTION

### Preparation of Botanically Derived Purified CBD

The following describes the production of the botanically derived purified CBD which comprises greater than or equal to 98% w/w CBD and less than or equal to other cannabinoids was used in the open label, expanded-access program described in Example 1 below.

In summary the drug substance used in the trials is a liquid carbon dioxide extract of high-CBD containing chemotypes of Cannabis sativa L. which had been further purified by a solvent crystallization method to yield CBD. The crystallisation process specifically removes other cannabinoids and plant components to yield greater than 95% CBD w/w, typically greater than 98% w/w.

The Cannabis sativa L. plants are grown, harvested, and processed to produce a botanical extract (intermediate) and then purified by crystallization to yield the CBD (botanically derived purified CBD).

The plant starting material is referred to as Botanical Raw Material (BRM); the botanical extract is the intermediate; and the active pharmaceutical ingredient (API) is CBD, the drug substance.

All parts of the process are controlled by specifications. The botanical raw material specification is described in Table A and the CBD API is described in Table B.

TABLE A
CBD botanical raw material specification

| Test | Method | Specification |
|---|---|---|
| Identification: | Visual | Complies |
| -A | TLC | Corresponds to standard |
| -B | | (for CBD & CBDA) |
| -C | HPLC/UV | Positive for CBDA |
| Assay: | In-house | NLT 90% of assayed |
| CBDA + CBD | (HPLC/UV) | cannabinoids by peak area |
| Loss on Drying | Ph. Eur. | NMT 15% |
| Aflatoxin | UKAS method | NMT 4ppb |
| Microbial: | Ph. Eur. | NMT10⁷cfu/g |
| - TVC | | NMT10⁵cfu/g |
| - Fungi | | NMT10²cfu/g |
| - E.coli | | |
| Foreign Matter: | Ph. Eur. | NMT 2% |
| Residual Herbicides | Ph.Eur. | Complies |
| and Pesticides | | |

TABLE B
Specification of an exemplary botanically derived purified CBD preparation

| Test | Test Method | Limits |
|---|---|---|
| Appearance | Visual | Off-white / pale yellow crystals |
| Identification A | HPLC-UV | Retention time of major peak corresponds to |
| | | certified CBD Reference Standard |
| Identification B | GC-FID/MS | Retention time and mass spectrum of major |
| | | peak corresponds to certified CBD Reference |
| | | Standard |
| Identification C | FT-IR | Conforms to reference spectrum for certified |
| | | CBD Reference Standard |
| Identification D | Melting Point | 65 - 67° C. |
| Identification E | Specific Optical | Conforms with certified CBD Reference |
| | Rotation | Standard; −110° to −140° (in 95% ethanol) |
| Total Purity | Calculation | ≥98.0% |
| Chromatographic Purity 1 | HPLC-UV | ≥98.0% |
| Chromatographic Purity 2 | GC-FID/MS | ≥98.0 % |
| CBDA | HPLC-UV | NMT 0.15% w/w |
| CBDV | | NMT 1.0% w/w |
| THC | | NMT 0.1% w/w |
| CBD-C4 | | NMT 0.5% w/w |
| Residual Solvents: | GC | NMT 0.5% w/w |
| Alkane | | NMT 0.5% w/w |
| Ethanol | | |
| Residual Water | Karl Fischer | NMT 1.0% w/w |

The purity of the botanically derived purified CBD preparation was greater than or equal to 98%. The botanically derived purified CBD includes THC and other cannabinoids, e.g., CBDA, CBDV, CBD-C1, and CBD-C4.

Distinct chemotypes of the Cannabis sativa L. plant have been produced to maximize the output of the specific chemical constituents, the cannabinoids. Certain chemovars produce predominantly CBD. Only the (−)-trans isomer of CBD is believed to occur naturally. During purification, the stereochemistry of CBD is not affected.

### Production of CBD Botanical Drug Substance

An overview of the steps to produce a botanical extract, the intermediate, are as follows:
a) Growing
b) Direct drying
c) Decarboxylation
d) Extraction—using liquid CO₂
e) Winterization using ethanol
f) Filtration
g) Evaporation

High CBD chemovars were grown, harvested, dried, baled and stored in a dry room until required. The botanical raw material (BRM) was finely chopped using an Apex mill fitted with a 1 mm screen. The milled BRM was stored in a freezer prior to extraction.

Decarboxylation of CBDA to CBD was carried out using heat. BRM was decarboxylated at 115° C. for 60 minutes.

Extraction was performed using liquid CO₂ to produce botanical drug substance (BDS), which was then crystalized to produce the test material. The crude CBD BDS was winterized to refine the extract under standard conditions (2 volumes of ethanol at −20° C. for approximately 50 hours). The precipitated waxes were removed by filtration and the solvent was removed to yield the BDS.

### Production of Botanically Derived Purified CBD Preparation

The manufacturing steps to produce the botanically derived purified CBD preparation from BDS were as follows:
a) Crystallization using C₅-C₁₂ straight chain or branched alkane
b) Filtration
c) Vacuum drying

The BDS produced using the methodology above was dispersed in C₅-C₁₂ straight chain or branched alkane. The mixture was manually agitated to break up any lumps and the sealed container then placed in a freezer for approximately 48 hours. The crystals were isolated via vacuum filtration, washed with aliquots of cold C₅-C₁₂ straight chain or branched alkane, and dried under a vacuum of <10 mb at a temperature of 60° C. until dry. The botanically derived purified CBD preparation was stored in a freezer at −20° C. in a pharmaceutical grade stainless steel container, with FDA food grade approved silicone seal and clamps.

### Physicochemical Properties of the Botanically Derived Purified CBD

The botanically derived purified CBD used in the clinical trial described in the invention comprises greater than or equal to 98% (w/w) CBD and less than or equal to 2% (w/w) of other cannabinoids. The other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w).

The botanically derived purified CBD used additionally comprises a mixture of both trans-THC and cis-THC. It was found that the ratio of the trans-THC to cis-THC is altered and can be controlled by the processing and purification process, ranging from 3.3:1 (trans-THC:cis-THC) in its unrefined decarboxylated state to 0.8:1 (trans-THC:cis-THC) when highly purified.

Furthermore, the cis-THC found in botanically derived purified CBD is present as a mixture of both the (+)-cis-THC and the (−)-cis-THC isoforms.

Clearly a CBD preparation could be produced synthetically by producing a composition with duplicate components.

Example 1 below describes the use of a botanically derived purified CBD in an open label, expanded-access program to investigate the clinical efficacy and safety of purified pharmaceutical cannabidiol formulation (CBD) as an adjunctive therapy in refractory epileptic spasms (ES).

### EXAMPLE 1

### Clinical Efficacy and Safety of Purified Pharmaceutical Cannabidiol Formulation (Cbd) as an Adjunctive Therapy in Refractory Epileptic Spasms (Es).

### Study Design

Subjects were required to be on one or more AEDs at stable doses for a minimum of two weeks prior to baseline and to have stable vagus nerve stimulation (VNS) settings and ketogenic diet ratios for a minimum of four weeks prior to baseline.

Patients were administered botanically derived purified CBD in a 100 mg/mL sesame oil-based solution at an initial dose of five milligrams per kilogram per day (mg/kg/day) in two divided doses. Dose was then increased weekly by 5 mg/kg/day to a goal of 25 mg/kg/day.

A maximum dose of 50 mg/kg/day could be utilised for patients who were tolerating the medication but had not achieved seizure control; these patients had further weekly titration by 5 mg/kg/day.

There were nine patients in this study, and each received CBD for at least 12 months. Modifications were made to concomitant AEDs as per clinical indication.

Seizure frequency, intensity, and duration were recorded by caregivers in a diary during a baseline period of at least 28 days. Changes in seizure frequency relative to baseline were calculated after 2 weeks and 1, 2, 3, 4, 6, 9 and 12 months of treatment. Epileptic spasms were counted by clusters.

During subsequent follow-up visits, parents provided weekly logs regarding cognitive changes, behavioural changes, and adverse events while taking CBD. Additionally, subjects had electroencephalogram (EEG) testing prior to and after the initiation of CBD.

Characteristics of background, interictal and ictal states as well as sleep architecture were recorded. Changes in the background included any variation in frequency, asymmetry and pattern of slowing, organization and gradient. Changes in the interictal state included only variations in the morphology, prevalence, and focality of the epileptiform discharges (EDs). Ictal changes were assessed based on changes in the seizure count. Last, the sleep states were assessed based on changes in the sleep architecture.

### Statistical Methods:

Patients were defined as responders if they had more than 50% reduction in seizure frequency compared to baseline. Patients, who did not have a follow-up seizure count in a given month, were excluded from calculations in that corresponding month. The percent change in seizure frequency was calculated as follows:

$$\% change seizure frequency = \frac{\begin{matrix}( weekly seizure frequency Month x ) - \\ ( weekly seizure frequency Baseline )\end{matrix}}{( weekly seizure frequency Baseline )}$$

The percent change reported at each time interval reflects reported seizure frequency over the past month relative to baseline. The EEG findings were correlated to the corresponding month seizure frequency change as well as the baseline seizure frequency.

### Results

### Patient Description

The nine patients enrolled in the open label; expanded-access program had refractory ES in addition to other seizure types.

The average age was nine years (range: 2-16 years, median: 8 years), four were male and five were female. Spasm etiologies included tuberous sclerosis complex (n=3), Dup15q syndrome (n=1), bilateral cerebral dysgenesis (n=1), lissencephaly (n=1), and a CASK loss of function mutation (n=1) as detailed in Table 1 below.

TABLE 1
Patient demographics and concomitant medication

| | | | | Age of | Other | | Number |
|---|---|---|---|---|---|---|---|
| Patient | Age | | | Spasm onset | seizure | Concomitant | of Past |
| Number | (years) | Sex | Etiology | (months) | types | AEDs | AEDs |
| 1 | 14 | M | Dup15q13 | 6 | yes | VPA, LEV, | 11 |
| | | | | | | CLB | |
| 2 | 16 | M | Cerebral | 4 | yes | VGB, ZNS, | 9 |
| | | | dysgenesis | | | CLB | |
| 3 | 10 | F | Lissencephaly | 4 | yes | CLB, LEV, | 6 |
| | | | | | | RFN | |
| 4 | 5 | M | None | 9 | yes | LCS, CLB, | 7 |
| | | | | | | TPM, LEV | |
| 5 | 2 | F | None | 4 | no | TPM, VGB, | 4 |
| | | | | | | CLB | |
| 6 | 8 | M | TSC | 4 | yes | LCS, LEV, | 8 |
| | | | | | | CLB | |
| 7 | 13 | F | TSC | 6 | yes | LCS, CLB, | 9 |
| | | | | | | VPA | |
| 8 | 3 | F | TSC | 16 | yes | LEV, CLB | 6 |
| 9 | 8 | F | CASK LoF | 21 | yes | VGB, CLB, | 10 |
| | | | | | | LCS | |

VPA = valproic acid, LEV = levetiracetam, CLB = clobazam, VGB = vigabatrin, ZNS = zonisamide, RFN = rufinamide, LOS = lacosamide, TPM = topiramate, LTG = lamotrigine

The average age of ES onset was eight months (range: 4-21 months, median: 6 months).

### Study Medication and Concomitant Medications

All nine patients were titrated up to at least 25 mg/kg/day of CBD, eight patients were titrated up to at least 45 mg/kg/day (#1-8), and six patients were titrated up to 50 mg/kg/day (#1-4, 6, 7). Patients 3 and 6 reached a dose of 45 mg/kg/day and 50 mg/kg/day, respectively, but had to reduce the dose to 35 mg/kg/day due to diarrhoea.

Patients had tried an average of eight AEDs prior to enrolment (range: 4-11 AEDs, median: 8 AEDs). Seven (78%) patients had been on vigabatrin (VBG), four (57%) of whom (# 4, 6, 7, and 8) discontinued before initiation of CBD treatment due to lack of efficacy.

Additionally, five (56%) patients (#1, 4, 5, 6, and 9) had a course of ACTH before beginning CBD treatment all without significant benefit. The average number of concomitant AEDs at the time of starting CBD was three per patient (range: 2-4, median: 3) three patients had a functional VNS.

All patients were on clobazam (CLB) with doses ranging from 10-60mg at time of initiation of CBD. The dose of the CLB was lowered during the study period in eight of the nine patients and discontinued in one of the nine patients.

### Clinical Changes

The average baseline was 20.3 ES per week (range: 3.6-51.8 seizures per week median: 21.8 seizures per week). Tables 2 and 3 illustrate the percent change in seizure frequency for each patient as well as the average and mean responder rates (percent of patients with a greater than 50% reduction in spasm frequency).

TABLE 2
Percent Change in Spasm Frequency Over Time

| Patient | 2 | 1 | 2 | 3 | 6 | 9 | 12 |
|---|---|---|---|---|---|---|---|
| number | weeks | month | months | months | months | months | months |
| 1 | −74.50% | −84.20% | −34.70% | −21.80% | −75.70% | 80.10% | 163.10% |
| 2 | −100% | −92.50% | −82.50% | −60.00% | −67.50% | −85.00% | −85.00% |
| 3 | −100% | −100% | −100% | −100% | −100% | −100% | −100% |
| 4 | −100% | −96.00% | −100% | −100% | −100% | −100% | −100% |
| 5 | 10.60% | 10.60% | 9.60% | 12.30% | −29.80% | −76.20% | −42.40% |
| 6 | −95.30% | −94.20% | −75.60% | −74.40% | −80.20% | −100% | −100% |
| 7 | −98.80% | −92.50% | −100% | −100% | −100% | −100% | −100% |
| 8 | −15.10% | −3.70% | −58.30% | −49.10% | −92.20% | −85.80% | −100% |
| 9 | −34.40% | −52.40% | −35.70% | −38.30% | −17.60% | 13.13% | 2.07% |
| Average | −67.50% | −67.20% | −64.10% | −59.00% | −73.70% | −61.53% | −51.35% |
| Responder | 66.70% | 77.80% | 66.70% | 55.60% | 77.80% | 77.80% | 77.80% |
| Rate | | | | | | | |

TABLE 3
Percent Change in Spasm Frequency at Different CBD Doses

| Patient | 10 | 20 | 25 | 35 | 45 | 50 |
|---|---|---|---|---|---|---|
| number | mg/kg/day | mg/kg/day | mg/kg/day | mg/kg/day | mg/kg/day | mg/kg/day |
| 1 | −74.50% | −84.20% | −30.80% | 80.10% | N/A | 163.20% |
| 2 | −100% | −92.50% | −68.10% | N/A | N/A | −85.00% |
| 3 | −100% | −100% | −100% | −100% | −100% | −100% |
| 4 | −100% | −96.00% | −100% | −100% | −100% | −100% |
| 5 | 10.60% | 10.60% | 10.90% | −42.50% | −15.20% | N/A |
| 6 | −95.30% | −94.20% | −75.60% | −80.50% | N/A | −79.70% |
| 7 | −98.80% | −92.50% | −100% | −100% | −100% | −100% |
| 8 | −15.10% | −3.70% | −58.30% | −49.10% | −89.20% | N/A |
| 9 | −34.40% | −52.40% | −30.50% | N/A | N/A | N/A |
| Average | −67.50% | −67.20% | −61.40% | −56.00% | −80.90% | −50.20% |
| Responder rate | 66.70% | 77.80% | 66.70% | 57.10% | 80.00% | 83.30% |

The overall responder rates after 2 weeks, 1 month, 2 months, 3 months, 6 months, 9 months, and 12 months of treatment with CBD were 66.7%, 77.8%, 66.7%, 55.6%, 77.8%, 77.8 and 77.8%, respectively.

One patient was epileptic spasm-free after one month of CBD treatment and two other patients were epileptic spasm-free after two months of CBD treatment. After 12 months of treatment with CBD, five (55.6%) patients were spasm-free.

CBD was also effective in reducing the frequency of other seizure types experienced by the patients; one patient became seizure free of tonic-clonic seizures after starting CBD treatment. Additionally, in four (44%) patients, caregivers reported sustained improvements in alertness, verbal capacity and communications, cognitive availability, and initiation of emotional and physical connections.

### Conclusions

These data indicate that CBD was able to significantly reduce the number of epileptic spasms. Clearly the treatment is of significant benefit in this difficult to treat seizure type given the high responder rate experienced in all patients.

Of interest is that patients suffering from TSC (patients 6, 7 and 8) obtained significant benefit whereby all three were completely seizure free after 12 months of treatment.

In addition, the use of CBD in combination with clobazam in the treatment of epileptic spasms appears to provide significant benefit.

1-14. (canceled)

15. A method of treating a patient suffering from epileptic spasms (ES) comprising administering a cannabidiol (CBD) preparation to the subject in need thereof

16. The method according to claim 15, wherein the CBD preparation comprises greater than or equal to 98% (w/w) CBD and less than or equal to 2% (w/w) other cannabinoids, wherein the less than or equal to 2% (w/w) other cannabinoids comprise the cannabinoids tetrahydrocannabinol (THC); cannabidiol-C1 (CBD-C1); cannabidivarin (CBDV); and cannabidiol-C4 (CBD-C4), and wherein the THC is present as a mixture of trans-THC and cis-THC.

17. The method according to claim 15, wherein the CBD preparation is used in combination with one or more concomitant anti-epileptic drugs (AED).

18. The method according to claim 17, wherein the one or more AED is selected from the group consisting of: valproic acid, levetiracetam, clobazam, vigabatrin, zonisamide, rufinamide, lacosamide, topiramate and lamotrigine.

19. The method according to claim 18, wherein the AED is clobazam.

20. The method according to claim 15, wherein the CBD is present is isolated from cannabis plant material.

21. The method according to claim 15, wherein at least a portion of at least one of the cannabinoids present in the CBD preparation is isolated from cannabis plant material.

22. The method according to claim 15, wherein the CBD is present as a synthetic preparation.

23. The method according to claim 22, wherein at least a portion of at least one of the cannabinoids present in the CBD preparation is prepared synthetically.

24. The method according to claim 15, wherein the dose of CBD is greater than 5 mg/kg/day.

25. The method according to claim 15, wherein the dose of CBD is 20 mg/kg/day.

26. The method according to claim 15, wherein the dose of CBD is 25 mg/kg/day.

27. The method according claim 15, wherein the dose of CBD is 50 mg/kg/day.

28. The method according to claim 15, wherein the epileptic spasms are associated with tuberous sclerosis complex (TSC).
